(19) United States
(12) Patent Application Publication
(10) Pub. No.: US 2025/0149114 A1
(43) Pub. Date: May 8, 2025

(54) METHODS AND SYSTEMS FOR DEFINING TEST SAMPLE READ COUNT LIMITS FOR A RANGE OF MICROBIAL SEQUENCE READS

(71) Applicant: DNA GENOTEK INC., Kanata, Ontario (CA)

(72) Inventors: Denise B. LYNCH, Ontario (CA); Evgueni Vladimirovitch DOUKHANINE, Ontario (CA)

(21) Appl. No.: 18/837,523

(22) PCT Filed: Feb. 9, 2023
(86) PCT No.: PCT/CA2023/050169

§ 371 Date: Aug. 9, 2024

Related U.S. Application Data
(60) Provisional application No. 63/308,665, filed on Feb. 10, 2022.
(60) Provisional application No. 63/325,357, filed on Mar. 30, 2022.

(51) Int. Cl. G16B 20/10; C12N 1/04

(52) U.S. Cl. CPC G16B 20/10; C12N 1/04

(57) ABSTRACT

Method and systems are provided for defining test sample read count limits for a range of microbial sequence reads. A computing device may generate a stabilization framework usable to determine a viability of a stabilization solution to stabilize a particular microbial species. The stabilization framework may include a first read count limit defined based on an enumerated abundance of the microbial species in a control sample multiplied by a first sequencing range value. The computing device may receive a read count value corresponding to an enumerated abundance of the microbial species in a mixture comprising a biological sample and the stabilization solution. The stabilization framework maybe applied to the read count value to generate an output based on whether the read count value exceeds the first read count limit. The computing device may then select, the stabilizing solution for use with subsequent biological samples.

### CROSS-REFERENCE TO RELATED APPLICATIONS

The present patent application claims the benefit of priority to U.S. Provisional Patent Application No. 63/308,665 filed Feb. 10, 2022, and to U.S. Provisional Patent Application No. 63/325,357 filed Mar. 30, 2022, which are both incorporated herein by reference in their entirety for all purposes

### TECHNICAL FIELD

This disclosure generally relates to defining deoxyribonucleic acid (DNA) read count limits in a sample; and more specifically to using defined DNA read count limits for various applications including the identification of suitable stabilization solutions for samples containing a particular microbial species.

### BACKGROUND

Biological samples containing microbial species may be subject to various tests based on the particular microbial species present in the biological sample, a quantity of the microbial species in the biological sample, etc. Preparation for some of these tests may include polymerase chain reaction (PCR) based amplification of the DNA of the particular microbial species. The PCR process may generate copies of the DNA of the particular microbial species within the biological sample enabling DNA analysis of the microbial species within the biological sample. In some instances, PCR amplification may not provide viable test results for a biological sample. PCR biases may be introduced during amplification and may result in inaccurate indications of a quantity of the microbial species in the biological sample. PCR biases may be more likely to impact the amplification of lower-abundant microbial species in the biological sample than the amplification of higher-abundant microbial species. PCR biases may render some applications, such as those that may rely on a particular quantity of the microbial species being present, to be potentially inoperable. Even when those applications are technically operable, the high degree of variability between replicates may prevent a consistent application or an application with a predictable degree of success.

### SUMMARY

Methods and systems are described herein for defining test sample read count limits for a range of microbial sequence reads. The methods include: generating, by a computing device, a stabilization framework usable to determine a viability of a stabilization solution to stabilize a particular microbial species, the stabilization framework including a first read count limit defined based on an enumerated abundance of the microbial species in a control sample multiplied by a first sequencing range value; receiving a read count value corresponding to an enumerated abundance of the microbial species in a mixture comprising a biological sample and the stabilization solution; applying the stabilization framework to the read count value, wherein the stabilization framework generates an output based on whether the read count value exceeds the first read count limit; and selecting, in response to the output indicating that the read count value exceeds the first read count limit, the stabilizing solution for use with subsequent biological samples, wherein the biological sample and the subsequent biological samples are of a same type.

Systems are described herein for defining test sample read count limits for a range of microbial sequence reads. The systems include one or more processors and a non-transitory computer-readable medium storing instructions that, when executed by the one or more processors, cause the one or more processors to perform any of the methods as previously described.

Non-transitory computer-readable media are described herein for storing instructions that, when executed by the one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

FIG. 1 illustrates an example system in which Test Sample Read Count Limits may be defined for various applications according to aspects of the present disclosure.

FIG. 2 illustrates a graph depicting example Test Sample Read Count Limits for an example microbial species according to aspects of the present disclosure.

FIG. 3 illustrates a flowchart of an example process for applying Test Sample Read Count Limits to identify viable stabilization solutions according to aspects of the present disclosure.

FIG. 4 illustrates an example computing device architecture of a computing device that can implement the various techniques described herein according to aspects of the present disclosure

### DETAILED DESCRIPTION

The present disclosure includes systems and methods for defining test sample read count limits (TeSaRCoLs) for particular microbial species and for various applications. Various tests applied to biological test samples may include PCR-based amplification of DNA associated with particular microbial species in the biological test sample. Some microbial species, such as lower-abundant microbial species, may be more likely to be impacted by PCR-biases causing inaccurate indications of microbial species abundance and a higher likelihood of variations between replicates. TeSaRCoLs can mitigate the impact of PCR-biases by providing threshold read counts usable to identify acceptable feature read counts of a microbial species. TeSaRCoLs may be applied to compare two paired samples, from a same source (e.g., a same donor over longitudinal sample collection, different donors with matched analyte source, etc.), after sequencing the nucleic acid pool and binning the sequenced reads by feature annotation (e.g., taxonomic, gene, another molecular marker, etc.) or after detection and enumeration of the molecular signal.

TeSaRCoLs may include an upper threshold that can be defined as u-TeSaRCoL=A*2^{(0.5+5/log}^{₁₀}^{(A))} and a lower threshold that can be defined as l-TeSaRCoL=A*2^{(0.5+5/log}^{₁₀}^{(A))}, where A corresponds to the feature read count of a particular microbial species in a baseline biological sample. The multiplier of 2 to a given power may represent a fold-change and the power can be positive or negative to capture the increase or decrease allowed for the test sample, respectively. In some applications, a desirable determination may correspond to a read count of a biological test sample that is within the TeSaRCoLs (e.g., a read count that is greater than l-TeSaRCoL and less than u-TeSaRCoL, etc.). For example, in some applications, test samples that fall within l-TeSaRCoL and u-TeSaRCoL may be preserved and/or the corresponding test results of such biological test samples may be preserved. The read counts of biological test samples that do not fall within l-TeSaRCoL and u-TeSaRCoL may be purged from further analysis in favor of another biological test sample that does fall within l-TeSaRCoL and u-TeSaRCoL. A test case sample that falls outside l-TeSaRCoL and u-TeSaRCoL may indicate that an impermissible change in sample composition has occurred between the control sample and the test sample.

In some examples, two biological test samples may be obtained (e.g., a control sample and a test sample). The upper read count limit of the baseline sample (A) may be calculated using the control sample to derive l-TeSaRCoL and u-TeSaRCoL for the two biological test samples. The test sample may then be tested to determine a degree of deviation from the control sample. In one application, the test sample may be exposed to one or more constraints (e.g., such as, but not limited to, time, one or more stabilization solutions, one or more drugs such as a prescribed treatment or otherwise, one or more microbial species, one or more viral agents, combinations thereof, etc.). The l-TeSaRCoL and u-TeSaRCoL may be used to determine an effect of the exposure on the test sample by measuring the deviation from the control sample. If the constraint is a stabilization solution, a degree of deviation (e.g., via l-TeSaRCoL and u-TeSaRCoL) of the test sample from the control sample can be determined. A deviation with the thresholds of l-TeSaRCoL and u-TeSaRCoL may be indicative of a viable stabilization solution that can be used to preserve the biological sample test sample (and/or an analogous biological test sample obtained via similar conditions) for at least the predetermined time interval.

In other applications, a desirable determination may correspond to a feature read count of the test sample that is outside the thresholds of l-TeSaRCoL and u-TeSaRCoL. For example, a biological test sample obtained from the gut of a human patient (e.g., such as a fecal sample, etc.), may be sequenced to determine a degree of absorption of an administered therapeutic (or lack thereof). The control sample may be obtained at an initial or pretreatment time point and used to derive l-TeSaRCoL and u-TeSaRCoL (as previously described). The test sample may be obtained at a predetermined time interval after the control sample. The feature read count of the test sample may be compared to the l-TeSaRCoL and u-TeSaRCoL to determine the degree of deviation. A large deviation (e.g., such as when the feature read count is greater than u-TeSaRCoL or less than l-TeSaRCoL, etc.) may be indicative of a high degree of the therapeutic being absorbed. A small or no deviation between the control sample and the test sample (e.g., such as when the feature read count is less than u-TeSaRCoL and greater than l-TeSaRCoL, etc.) may be indicative of a low degree of therapeutic absorption. The thresholds defined by u-TeSaRCoL and l-TeSaRCoL indicate an efficacy of a current therapeutic course of action, which may be used to define new therapeutic courses of action (e.g., such as increase in dosage of current therapeutic when detecting low degree of therapeutic absorption, new therapeutics when detecting a low degree of therapeutic absorption, terminating an administration of the current therapeutic when detecting a high degree of absorption, etc.).

Applications of TeSaRCoL may use an input that corresponds to a counts table of number of reads per feature of a particular microbial species, per sample generated from high-throughput sequencing of a biological sample source, and/or after bioinformatic quality control and interpretation. The bounds of an excepted sample variability may be empirically determined using internal features (markers) of interest. These markers can be generalizable to the whole sample composition and provide a measurable indication of change outside the accepted bounds of variability. TeSaRCols may be operable for both low and high abundances features. TeSaRCols may also be applicable to any high-throughput sequencing or molecular output (including, but not limited to, targeted or untargeted output, nucleic acid sequencing, metabolic profiling, volatile compound measurement etc.) with appropriately chosen and tested internal markers representing the variability in the specific context of sample and method type.

Applications of TeSaRCols can include, but are not limited to identifying sample integrity, identifying sample stability, identifying treatment efficacy, combining TeSaRCols with controlled sample inputs or spike-ins, and/or the like. Identifying test sample integrity may include a test that detects sample deviations due to time, tempernture, and/or other external intervening factors that may compromise the test sample integrity and/or alter the composition of a test sample relative to its baseline state. For example, TeSaRCols may be used to determine test sample integrity after a test sample is exposed to freezing temperatures for a predetermined time interval (e.g., seconds, minutes, hours, days, years, etc.) relative to a recently collected test sample from the same source or a test sample that lacks such exposure to freezing temperatures. Such a determination may be used as a quality control test to determine whether a stored sample remains viable for further or continued use (e.g., probiotic mixture, fecal transplant material, multi-lab processing and transport, etc.).

Identifying sample stability may correspond to a test that detects a deviation in sample composition. A biological and/or in vitro sample may be collected before a treatment and after a treatment and compared via TeSaRCols to determine whether the later-collected sample deviates from the earlier-collected sample indicating a deviation in sample composition. Identifying sample stability may include an in vitro cell line, mock community, single or multi organism culture that has been sampled before and after an exogenous treatment (e.g., drug, antibiotic, nutrient, environmental stressor, etc.). Identifying that the later-collected sample deviates from the earlier-collected sample relative to TeSaRCols may support claims regarding microbiome impacts of a particular drug, nutrient, diet regimen, supplement, or other course of treatment. The test and/or the test results may be used to determine that a treatment has altered one or more intended target(s) (e.g., microbial species, etc.), measure the extent in which the treatment has altered the one or more intended target(s), and/or measure any off-target effects of the treatment.

Identifying treatment efficacy may correspond to a test that detects deviation in sample composition between samples obtained from an individual before and after treatment for a disease or illness. In an illustrative example, such a test may be performed using biological samples obtained from an individual with a Clostridium difficile infection, inflammatory bowel disease (IBD), or the like, before and after treatment with one or more antibiotics, before and after single treatment or multiple treatments with a fecal matter transplant, etc. The earlier-collected samples may be compared to the later-collected samples to determine whether a treatment has altered one or more intended targets, measure a dose dependent effect on a particular microbial composition, and/or the like, which may directly relate to the disease state of the individual. Identifying treatment efficacy may be operable using biological samples obtained from individuals with infections (e.g., bacterial, viral, and/or fungal), other diseases, and/or illness.

Combining TeSaRCols with controlled sample inputs or spike-ins, and/or the like may correspond to one or more tests that measure performance of an assay in repeated processing or interoperability between two or more locations, devices, and/or protocols performing a same assay or measurement of the same device over a predetermined time interval. The test may be used to assess a lack of change in performance/specification of the assay. The test may include comparing the processes and/or results of one lab performing sequencing of microbiome samples to another lab performing sequencing of the same or similar microbiome samples. Alternatively, or additionally, the test may include comparing the processes or results of one lab performing sequencing of microbiome samples to the same lab across devices and/or protocols. The information derived from such a test may be used to qualify a laboratory, service, device, protocol, and/or the like for execution, application, and/or use of a particular laboratory, service, device, protocol, and/or the like.

In an illustrative example, methods and systems may be provided for determining an acceptable read count of a biological test sample for one or more applications. A computing device may generate a framework that includes thresholds configured to identify one or more acceptable read counts associated with a particular microbial species. For example, the framework may include a first read count limit defined based on an enumerated abundance of the particular microbial species in a control sample multiplied by a first sequencing range value. The first sequencing range value may include a fold-change value raised to a predetermined power that is based on the enumerated abundance of the particular microbial species in the control sample. In some examples, the fold-change value may be approximately 2 and the predetermined power may be positive and equal to approximately

$$( 0.5 + \frac{5}{{log}_{10} ( A )} ) ,$$

where A corresponds to the enumerated abundance of the particular microbial species. The framework may include a second read count limit based on an enumerated abundance of the particular microbial species in a control sample multiplied by a second sequencing range value. The second sequencing value corresponds to the fold-change raised to the negative of the predetermined power. The first read count limit may correspond to an upper read count limit and the second read count limit may correspond to a lower read count limit. For example, the first read count limit may be derived as

$$u − TeSaRCoL = A ⋆ {2}^{( 0.5 + \frac{5}{{log}_{10} ( A )} )}$$

and the second read count limit may be derived as

$$l − TeSaRCoL = A ⋆ {2}^{- ( 0.5 + \frac{5}{{log}_{10} ( A )} )} .$$

The control sample may be obtained at an initial time point and sequenced to determine the enumerated abundance of the particular microbial species within a predetermined time interval from the initial time point to avoid the time interval between obtaining the control sample and sequencing the control sample from affecting application in which time may affect the sample testing or any of the one or more applications.

In some instances, the framework may include a single threshold (e.g., just the first read count limit or the second read count limit, etc.), three or more thresholds, etc. A single threshold may be usable when the framework is determining a viable test sample as one that deviates from the control sample only in a particular direction. For example, test samples with a read count that is less than the first read count limit (e.g., the upper read count limit as previously described), may be viable for some applications of TeSaRCols. A deviation from the control sample that is lower than the first read count limit may still be viable. Multiple thresholds may be usable when the framework is determining viability of a test sample according to two or more applications. For example, a third read count limit may be defined as

$$3 - TeSaRCoL = \frac{1}{2} ( \frac{1}{2} ( A ⋆ {2}^{( 0.5 + \frac{5}{{log}_{10} ( A )} )} + A ⋆ {2}^{− ( 0.5 + \frac{5}{{log}_{10} ( A )} )} ) + A ⋆ {2}^{( 0.5 + \frac{5}{{log}_{10} ( A )} )} )$$

and a fourth read count limit may be defined as

$$4 - TeSaRCoL = \frac{1}{2} ( \frac{1}{2} ( A * {2}^{( 0.5 + \frac{5}{{log}_{10} ( A )} )} + A * {2}^{- ( 0.5 + \frac{5}{{log}_{10} ( A )} )} ) + A * {2}^{- ( 0.5 + \frac{5}{{log}_{10} ( A )} )} ) .$$

A read count value of a test sample that is less than the third read count value and greater than the fourth read count value may indicate a minor deviation in the composition of the test sample from the control sample. As a result, any test being performed by the test sample (e.g., a particular stabilization solution, therapeutic, etc.) may indicate viability for a first application. For example, a test sample including a particular stabilization solution having a read count with such a minor deviation from the control sample may be indicative of viable stabilization solutions for future biological samples including the particular microbial species. A read count value of the test sample that is greater than the third read count limit, but less than the first read count limit or greater than the second read count limit, but less than the fourth read count limit, may indicate a greater deviation (than the preceding example) in the composition of the test sample from the control sample. As a result, any test being performed by the test sample (e.g., a particular stabilization solution, therapeutic, etc.), may indicate that the test condition (e.g., the particular stabilization solution being tested, etc.) is not viable for the first application, but may be viable for the second application. A read count limit that is greater than the first read count limit or less than the second read count limit may not be viable for the first or the second application but may be viable for a third application, etc. The framework may define any number of the read count limits with each read count limit being defined based on the first read count limit and/or the second read count limit.

The computing device may receive a read count value corresponding to an enumerated abundance of the microbial species in a mixture comprising a biological sample. In some instances, the mixture may also include one or more solutions under test (e.g., stabilization solutions, etc.). The biological sample may be obtained from a same source (e.g., a same donor over longitudinal sample collection, different donors with matched analyte source, etc.) as the control sample. The solution may be a particular solution under test for viability in subsequently obtained biological samples. In some instances, multiple mixtures may be obtained each including a biological sample (from the same source as the control sample) and each including a different solution under test to determine a particular solution to use in subsequently obtained biological samples.

For example, the mixture may include a particular stabilization solution configured to stabilize the biological sample (or at least the particular microbial species) for a particular time interval and/or under particular environmental conditions (e.g., temperature, humidity, pressure, radiation, particular periodic elements, etc.).

The computing device may apply the framework to the read count value by comparing the read count value to the first read count limit, the second read count limit (and/or other read count limits if used), etc. The framework may generate an output indicative of whether the read count value is less than the first read count value and/or greater than the second read count value (if used). In addition, if the framework includes additional read count limits, the output may indicate whether the read count value is greater than or less than any of those read count limits. In some instances, the output may also include an indication of the degree in which the read count value differs from the first read count limit, the second read count limit, (and/or any of the additional read count limits, if used), etc. The output is indicative of a degree in which the composition of the mixture deviated from the control sample.

The degree in which the composition of the test sample deviates from the control sample may be usable based on one or more applications. For example, when selecting stabilization solutions, a high degree of deviation (e.g., such as a deviation in which the read count value is greater than the first or upper read count limit or less than the second or lower read count limit), may indicate that the stabilization solution in the mixture had little or no effect on maintaining the integrity or relative composition of the microbial species within the biological sample. A low degree of deviation (e.g., such as a deviation in which the read count value is less than the first read count limit and greater than the second read count limit) may indicate that the stabilization solution in the mixture adequately maintained the composition of the biological sample over the particular time interval and/or under particular environmental conditions.

If the computing device is testing for the viability of a stabilization solution (or other solution intended to not be affected by time and/or environmental factors), a low degree of deviation from the control sample may cause the computing device to select the stabilization solution (and/or other solution) for addition to subsequently obtained biological samples. If the framework output indicates a high degree of variability, then then the computing device may not select the particular stabilization solution (and/or other solution) for use in subsequently collected biological samples. In some instances, the computing device may select a new stabilization solution (and/or other solution) for use in a subsequent read count value test to determine the viability of the new stabilization solution (and/or other solution).

If the computing device is testing for other characteristics of the source individual and/or the particular microbial species (e.g., such as identifying sample integrity, identifying sample stability, identifying treatment efficacy, combining TeSaRCols with controlled sample inputs or spike-ins, etc.), the computing device may determine if a desirable output is one in which the read count value is greater than the first read count limit, less than the first read count limit and greater than the second read count limit, or less than the second read count limit. The computing device may then execute an action based on the determination of whether the output was desirable or not. For example, the test sample was obtained from an individual some time interval after administration of an antibiotic treatment, then an output indicating high degree of deviation (e.g., where the read count limit is greater than the first read count limit or less than the second read count limit) may be indicative that that the antibiotic was effective. If the output indicated a low degree of deviation (e.g., indicative that the antibiotic treatment may not have been effective), then the computing device may recommend a new treatment (e.g., such as a new antibiotic, etc.), recommend additional testing, and/or the like.

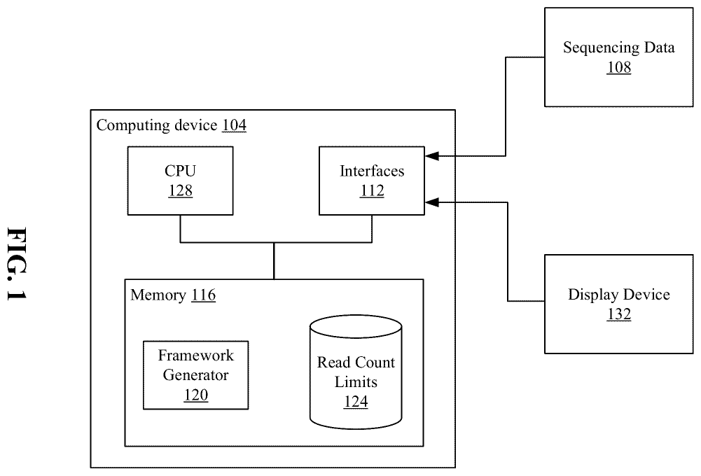

FIG. 1 illustrates an example system in which Test Sample Read Count Limits may be defined for various applications according to aspects of the present disclosure. Computing device 104 may receive sequencing data 108 corresponding to the sequencing of one or more microbial species of biological samples. The process of sequencing microbial species may include polymerase chain reaction (PCR) based amplifications of DNA molecules. PCR-biases may impact the sequencing data 108, which may corrupt the sequencing data and prevent the reproducibility of subsequent test results (e.g., by introducing a high degree of variability between replicates, etc.). Computing device 108 may apply TeSaRCoL to mitigate the impact of PCR-bias on test samples.

Computing device 104 may receive sequencing data 108 through interface 112. Interface 112 may include one or more input/output interfaces, communication interfaces, application programming interfaces (APIs), network interfaces, etc. that enable receiving and/or to components computing device 104 and/or external devices. Interfaces 116 may receive sequencing data 108, parse the incoming data stream, and store the parsed sequencing data 108 in memory 116. Memory 116 may include a framework generator usable to process sequencing data associated with pairs of biological samples obtained from a same source (e.g., a same donor over longitudinal sample collection, different donors with matched analyte source, etc.). The pairs of biological samples may include a control or baseline sample and a test sample.

Framework generator 120 may use the sequencing data associated with the control or baseline sample to define one or more Test Sample Read Count Limits (TeSaRCoL) for a particular microbial species. TeSaRCoL may be based on an enumerated abundance of the microbial species in a control sample. For example, an upper read count limit may be derived using

$$u - TeSaRCoL = A * {2}^{( 0.5 + \frac{5}{{log}_{10} ( A )} )}$$

and a lower read count limit may be derived using

$$l - TeSaRCoL = A * {2}^{- ( 0.5 + \frac{5}{{log}_{10} ( A )} )} .$$

The upper and lower read count may represent thresholds. TeSaRCoL may be used to determine a degree in which the microbial composition of a test sample deviates from the control or base line sample. A corresponding description of TeSaRCoLs can be found in U.S. Provisional Patent Application 63/308,665, which is hereby incorporated by reference in its entirety for all purposes.

In some instances, a framework generated by framework generator 120 may use TeSaRCoL to qualify samples for use in further testing. For example, the framework may use u-TeSaRCoL and l-TeSaRCoL to determine whether the composition of test sample has deviated from the control sample by a large enough margin so as to impact the accuracy of any subsequent testing. In those instances, a test sample may be viable when the test sample has a classified read count for a given microbial species that falls into the range set by the upper and lower TeSaRCols relative to the baseline sample. Test samples with read counts that are outside this range (e.g., having a read count value greater than u-TeSaRCoL or less than 1-TeSaRCoL, etc.) may be considered to have failed the test conditions.

A testable sample may be a sample including a particular microbial species having a read count that is above an established lower limit of quantitation (LLoQ) for that species and/or when the read count is within the lower bound of acceptance range for the test being applied. A lower read count minimum (e.g., lower baseline read count limit or l-BRCL) may be set for a baseline sample per microbial species being tested. The lower baseline read count limit may be defined as

$$l - BRCL = LLoQ - 1 = A * {2}^{- ( 0.5 + \frac{5}{{log}_{10} ( A )} )} ,$$

where A is the “lower baseline read count limit” (I-BRCL) to be calculated. Under some applications, a test sample with a read count that is at or below the LLoQ may be designated “failed”, as it will be outside of the acceptable read count range for the given test.

An upper baseline read count limit (or u-BRCL) can be derived based on an upper limit of quantitation (ULoQ). The upper baseline read count limit may be defined as

$$u - BRCL = ULoQ - 1 = A * {2}^{( 0.5 + \frac{5}{{log}_{10} ( A )} )} ,$$

where A is the “upper baseline read count limit” (u-BRCL) to be calculated. In some applications, a test sample with a read count that is at or above the ULoQ may be designated “failed”, as it will be outside of the acceptable read count range for the given test.

Frameworks may include executable instructions that derive u-TeSaRCoL and l-TeSaRCoL for a particular microbial species from sequencing data 108, executable instructions that compare a read count of a test sample to the u-TeSaRCoL and l-TeSaRCoL to determine viability of a test sample and/or for any of the previously disclosed applications, executable instructions that generate an output presenting a result of a test sample relative to the control or base line sample and based on u-TeSaRCoL and l-TeSaRCoL and/or the like. Generated frameworks may be stored in read count limits 124 database. In some instances, frameworks may be a self-contained set of executable instructions that once generated may execute on any hardware platform. For example, a framework generated by framework generator 120 may transmitted to another computing device for execution.

Frameworks may be generated based on a particular application of the read count limits. Some applications of the read count limits may include identifying how substances added to the test sample, environmental conditions exposed to the test sample, time, and/or the like affect the test sample. For those applications, the framework may be configured to define an acceptable range as being between the u-TeSaRCoL and l-TeSaRCoL. In other applications, in the acceptable range may be defined as being outside u-TeSaRCoL and l-TeSaRCoL so as to identify test samples that deviate substantially from the corresponding control or baseline samples.

Computing device 104 may receive sequencing data that corresponds to a particular test sample and identify the framework generated from the control and/or baseline sample that corresponds to the particular test sample. Test samples may be obtained from the same source as the control and/or baseline samples at a predetermined time interval after the control or baseline samples are collected and/or after the test sample is exposed to one or more environmental conditions (e.g., such as after the test sample traveled from a collection location to a testing location, etc.). The instructions of the framework may be executed by CPU 128 of computing device 104. The executed framework may determine whether the read count value is within the acceptable range (e.g., less than u-TeSaRCoL and greater than l-TeSaRCoL). In some instances, the framework may be configured such that the acceptable range is greater than u-TeSaRCoL and less than l-TeSaRCoL (e.g., such as determining an antibiotic treatment efficacy, etc.).

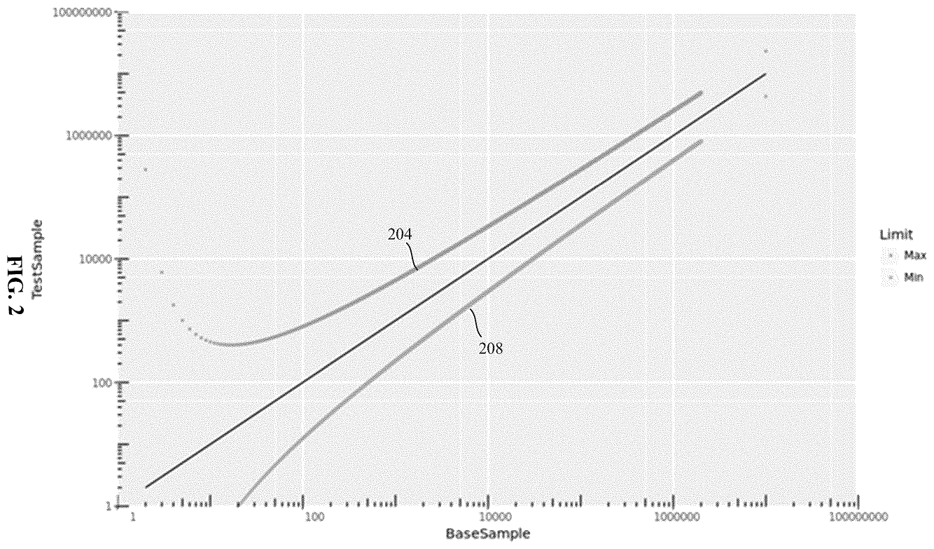

Computing device 104 may present the results of executing the framework using the read count of a test sample via a graphical user interface of display device 132. The presentation may include an indication of whether the read count of the test sample is within the acceptable range, a degree in which the read count deviates from the acceptable range and/or u-TeSaRCoL or l-TeSaRCoL, and/or the like. The presentation may also include other information associated with the test sample, the control and/or baseline samples, etc. such as, but not limited, an identification of one or more tests being run, an identification of a source of the test sample, an identification of conditions (e.g., environmental, time, etc.) exposed to the test sample, a timestamp corresponding to when the test sample was obtained, a timestamp corresponding to when the control or baseline samples were obtained, etc. In some instances, the presentation may include a graph (e.g., such as the graph of FIG. 2), where the read count limit of the test sample is plotted with u-TeSaRCoL and l-TeSaRCoL.

FIG. 2 illustrates a graph depicting example Test Sample Read Count Limits for an example microbial species according to aspects of the present disclosure. For each baseline sample (e.g., represented by the number of reads on the x-axis), a corresponding test sample (e.g., represented by the number of reads on the y-axis) may have the same quantity of reads (x=y line, in black). The upper line 204 may represent the upper read count limit u-TeSaRCoL and the lower line 208 may represent the lower read count limit l-TeSaRCoL. The upper line 204 and lower line 208 show a greater amount of variation allowed for lower abundant microbes (e.g., modelling after the observed increase in experimental and technical variation as relative baseline abundance of a given taxon within a sample goes down).

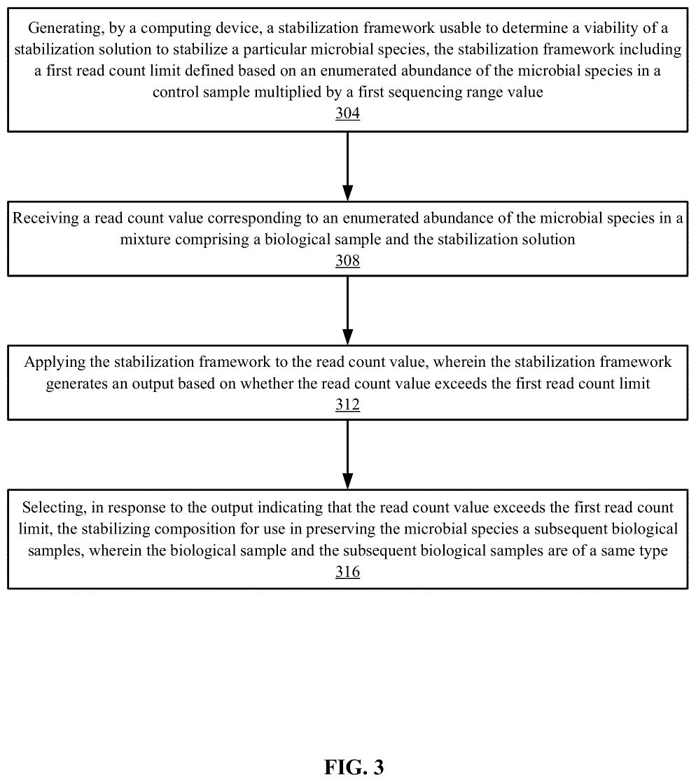

FIG. 3 illustrates a flowchart of an example process for applying Test Sample Read Count Limits to identify viable stabilization solutions according to aspects of the present disclosure. At block 304, a computing device generates a stabilization framework usable to determine a viability of a stabilization solution to stabilize a particular microbial species within a biological sample. The stabilization framework may represent a snapshot of the compositional characteristics of the microbiome of a control sample. The stabilization framework may be used to determine whether one or more microbial species in a test sample have diminished and/or multiplied relative to the control sample as determined by read count values. The stabilization framework may include one or more read count thresholds configured to identify one or more acceptable read counts associated with the particular microbial species. For example, the stabilization framework may include a first read count limit defined based on an enumerated abundance of the microbial species in a control sample multiplied by a first sequencing range value. The first sequencing range value may correspond to a fold-change value raised to a predetermined power that is based on the enumerated abundance of the particular microbial species in the control sample. In some examples, the fold-change value may be approximately 2 and the predetermined power may be positive or negative to capture an increase or decrease allowed for a test sample, respectively. The predetermined power may be equal to approximately

$$\pm ( 0.5 + \frac{5}{{log}_{10} ( A )} ) ,$$

where A corresponds to the enumerated abundance of the particular microbial species. For example, the first read count limit may be defined as

$$TeSaRCoL = A * {2}^{\pm ( 0.5 + \frac{5}{{log}_{10} ( A )} )} .$$

The control sample may be obtained at a first time. The control sample may be sequenced to determine the enumerated abundance of the particular microbial species within a predetermined time interval from the first time to avoid the time interval between obtaining the control sample and sequencing the control sample from affecting the determination of a viable stabilization solution.

In some instances, the stabilization framework may include two or more read count limits. In those instances, the stabilization framework may include a second read count limit based on an enumerated abundance of the particular microbial species in a control sample multiplied by a second sequencing range value. The second sequencing value may correspond to the fold-change raised to the predetermined power. If the stabilization framework includes a second read count limit, the predetermined power of the first read count limit may be set to positive to form an upper read count limit and the predetermined power of the second read count limit may be set to negative to from a lower read count limit. In that instance, the first read count limit may be derived as

$$u - TeSaRCoL = A * {2}^{( 0.5 + \frac{5}{{log}_{10} ( A )} )}$$

and the second read count limit may be derived as

$$l - TeSaRCoL = A * {2}^{- ( 0.5 + \frac{5}{{log}_{10} ( A )} )} .$$

The stabilization framework may include other read count limits in addition to the first read count limit and/or the second read count limit. The additional read count limits may be usable to test for additional stabilization solutions usable in particular applications. For example, a stabilization framework may be applied to a pair of biological samples (e.g., a control sample and corresponding test sample both obtained from a same source, etc.) to determine two or more applications of the stabilization solutions. A first application may necessitate a very small deviation in the read counts between the test sample and the control sample and second application may be permissive of some deviation in read counts between the test sample and the control sample. The stabilization framework may include additional read count limits based on a degree of deviation acceptable for a particular application of a stabilization solution. For example, a third read count limit may be defined as

$$3 - TeSaRCoL = \frac{1}{2} ( \frac{1}{2} ( A * {2}^{( 0.5 + \frac{5}{{log}_{10} ( A )} )} + A * {2}^{- ( 0.5 + \frac{5}{{log}_{10} ( A )} )} ) + A * {2}^{- ( 0.5 + \frac{5}{{log}_{10} ( A )} )} ) .$$

and a fourth read count limit may be defined as

$$4 - TeSaRCoL = \frac{1}{2} ( \frac{1}{2} ( A * {2}^{( 0.5 + \frac{5}{{log}_{10} ( A )} )} + A * {2}^{- ( 0.5 + \frac{5}{{log}_{10} ( A )} )} ) + A * {2}^{- ( 0.5 + \frac{5}{{log}_{10} ( A )} )} ) .$$

Other read count limits may be defined in addition those read count limits previously described. Those read count limits may be defined based on u-TeSaRCoL and/or l-TeSaRCoL.

At block 308, the computing device may receive a read count value corresponding to an enumerated abundance of the microbial species in a mixture comprising a biological sample and a stabilization solution under test. The biological sample may be obtained from the same source (e.g., same donor over longitudinal sample collection, different donors with matched analyte source, etc.) as the control sample. The stabilization solution under test may be selected from among multiple stabilization solutions to determine a viable stabilization solution for subsequently obtained biological samples containing the particular microbial species. In some instances, stabilization solution under test may be selected based on an expected time interval over which the test sample is expected to be stored before being tested and/or one or more expected environmental conditions (e.g., temperature, pressure, radiation, other microbial species, one or more viral species, one or more fungal species, and/or the like) that the test may be exposed to between being collected and being tested. In those instances, the biological sample may be obtained at some time interval after the control sample is obtained and/or after the mixture is exposed to one or more environmental conditions.

In some instances, multiple mixtures may be obtained, each including a biological sample (from the same source as the control sample) and each including a different stabilization solution selected from the multiple stabilization solutions to determine a particular solution use in subsequently obtained biological samples.

At block 312, the computing device may apply the stabilization framework to the read count value. The stabilization framework may output an indication as to whether the read count value exceeds the first read count limit. The stabilization framework may output an indication that the read count value exceeds the first read count limit when the read count value is greater than the first read count limit (and the predetermined power of the first read count limit is positive) or when the read count value is less than the first read count limit (and the predetermined power of the first read count limit is negative).

The stabilization framework may compare the read count value to a read count of the control sample to determine a degree in which the composition of the particular microbial species deviates from the control sample. The stabilization framework may generate an output that indicates whether the read count value is greater than the first read count limit, the difference between the read count value of the mixture and a read count value derived from control sample, a difference between the read count value and the first read count limit, and/or the like. If additional read count limits are used, the output may also indicate whether the read count value is greater than, equal to, or less than each read count limit and the difference between the read count value and each read count limit. For example, if a first read count limit and a second read count limit are used, the first read count limit may represent an upper read count limit and the second read count limit may represent a lower read count limit. The output may include an indication as to whether the read count value is greater than the first read count limit, between the first read count limit and the second read count limit, or less than the second read count limit in addition to the difference between the read count value and the closest read count limit to the read count value (or alternatively the difference between the read count value and each read count limit).

In some instances, the stabilization framework may indicate whether the stabilization solution under test passed or failed the test. For example, a failing designation may be assigned to a stabilization solution under test in which the read count value is greater than the first read count limit (and the predetermined power of the first read count limit is positive) or the read count value is less than the first read count limit (and the predetermined power of the first read count limit is negative). If additional read count limits are used such as the first (or upper) read count limit and a second (or lower) read count limit, then a failing designation may be assigned to a stabilization solution under test in which the read count value is greater than the first read count limit or less than the second read count limit. A failing designation may be indicative of a stabilization solution that failed to stabilize the biological sample (or the particular microbial species) by failing to prevent a substantial change (e.g., as determined by the read count limits) in the composition of the biological sample between the mixture and the control sample.

A passing designation may be assigned to a stabilization solution under test in which the read count value is less than the first read count limit (and the predetermined power of the first read count limit is positive) or the read count value is greater than the first read count limit (and the predetermined power of the first read count limit is negative). If additional read count limits are used such as the first (or upper) read count limit and a second (or lower) read count limit, then a passing designation may be assigned to a stabilization solution under test in which the read count value is less than the first read count limit and greater than the second read count limit. A pass designation may be indicative of a stabilization solution that is capable of stabilizing the biological sample (or the particular microbial species) by preventing a substantial change (e.g., as determined by the read count limits) in the composition of the biological sample between the mixture and the control sample.

At block 316, the computing device may select the stabilization solution in response to the read count the stabilization framework outputting the indication that the read count value exceeds the first read count limit. The selected stabilization solution may be used with subsequently obtained biological samples to stabilize the subsequently obtained biological samples. The biological sample and the subsequently obtained biological samples may be of a same type (e.g., including the particular microbial species, from a same source, from similar sources, etc.).

If the computing device is testing multiple stabilization solutions, the computing device may determine the best stabilization solution for use in stabilizing subsequently obtained biological sample. The computing device may test a set of stabilization solutions and first select the one or more stabilization solutions that receive a passing designation. The computing device may then determine which stabilization solution of the set of stabilization solutions caused the smallest deviation in the composition of the biological sample between the control sample and the mixture. For example, the stabilization solution that caused the smallest deviation in the composition of the biological sample may correspond to the stabilization solution in the mixture having a read count value closest to the read count value of the corresponding control sample.

In some instance, the computing device may include instructions that may automatically implement the selected stabilization solution. For example, the computing device may control (and/or be connected to a device that controls) the operations of a fabrication facility configured to manufacture biological sample collection devices. The computing device may transmit instructions (e.g., through one or more application programming interfaces, input/output interfaces, network interfaces, communication interfaces, etc.) directing the fabrication facility to include the selected stabilization solution in the manufacture of future biological sample collection devices.

Biological sample collection devices may be used in the collection and transport of biological samples. The biological sample collection device may prevent or limit environmental conditions from impacting the integrity and/or composition of the biological sample while the biological sample collection device is being transported from a collection site to a testing facility (e.g., laboratory, clinic, hospital, etc.). The biological sample may contact and/or mix with the stabilization solution within the biological sample collection device to stabilize the biological sample against time and/or one or more environmental conditions expected to be encountered during transit.

The first read count limit, second read count limit, and/or other read count limits may be usable to determine a degree of compositional deviation between the mixture (e.g., test sample) and the corresponding control sample. Such as determination may have other applications such as, but not limited to, identifying sample integrity, identifying sample stability, identifying treatment efficacy, combining TeSaRCoLs with controlled sample inputs or spike-ins, and/or the like as previously described.

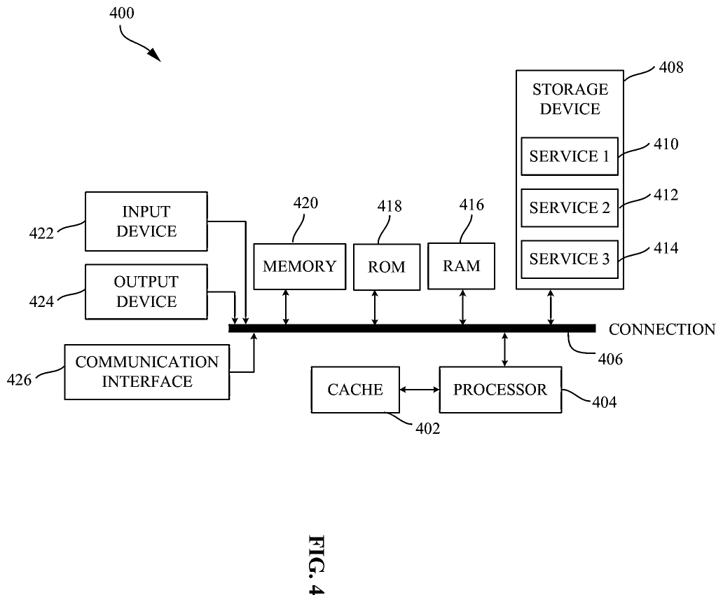

FIG. 4 illustrates an example computing system architecture including various components in electrical communication with each other and configured to implement aspects of the present disclosure. FIG. 4 illustrates a computing system architecture 400 including various components in electrical communication with each other using a connection 406, such as a bus, in accordance with some implementations. Example system architecture 400 includes a processing unit (CPU or processor) 404 and a system connection 406 that couples various system components including the system memory 420, such as ROM 418 and RAM 416, to the processor 404. The system architecture 400 can include a cache 402 of high-speed memory connected directly with, in close proximity to, or integrated as part of the processor 404. The system architecture 400 can copy data from the memory 420 and/or the storage device 408 to the cache 402 for quick access by the processor 404. In this way, the cache can provide a performance boost that avoids processor 404 delays while waiting for data. These and other modules can control or be configured to control the processor 404 to perform various actions.

Other system memory 420 may be available for use as well. The memory 420 can include multiple different types of memory with different performance characteristics. The processor 404 can include any general-purpose processor and a hardware or software service, such as service 1 410, service 2 412, and service 3 414 stored in storage device 408, configured to control the processor 404 as well as a special-purpose processor where software instructions are incorporated into the actual processor design. The processor 404 may be a completely self-contained computing system, containing multiple cores or processors, a bus, memory controller, cache, etc. A multi-core processor may be symmetric or asymmetric.

To enable user interaction with the computing system architecture 400, an input device 422 can represent any number of input mechanisms, such as a microphone for speech, a touch-sensitive screen for gesture or graphical input, keyboard, mouse, motion input, speech and so forth. An output device 424 can also be one or more of a number of output mechanisms known to those of skill in the art. In some instances, multimodal systems can enable a user to provide multiple types of input to communicate with the computing system architecture 400. The communications interface 426 can generally govern and manage the user input and system output. There is no restriction on operating on any particular hardware arrangement and therefore the basic features here may easily be substituted for improved hardware or firmware arrangements as they are developed.

Storage device 408 is a non-volatile memory and can be a hard disk or other types of computer readable media which can store data that are accessible by a computer, such as magnetic cassettes, flash memory cards, solid state memory devices, digital versatile disks, cartridges, RAMs 416. ROM 418, and hybrids thereof.

The storage device 408 can include services 410, 412, 414 for controlling the processor 404. Other hardware or software modules are contemplated. The storage device 408 can be connected to the system connection 406. In one aspect, a hardware module that performs a particular function can include the software component stored in a computer-readable medium in connection with the necessary hardware components, such as the processor 404, connection 406, output device 424, and so forth, to carry out the function.

The following examples illustrate various aspects of the present disclosure. As used below, any reference to a series of examples is to be understood as a reference to each of those examples disjunctively (e.g., “Examples 1-4” is to be understood as “Examples 1, 2, 3, or 4”).

Example 1 is a computer-implemented method comprising: generating, by a computing device, a stabilization framework usable to determine a viability of a stabilization solution to stabilize a particular microbial species, the stabilization framework including a first read count limit defined based on an enumerated abundance of the microbial species in a control sample multiplied by a first sequencing range value; receiving a read count value corresponding to an enumerated abundance of the microbial species in a mixture comprising a biological sample and the stabilization solution; applying the stabilization framework to the read count value, wherein the stabilization framework generates an output based on whether the read count value exceeds the first read count limit; and selecting, in response to the output indicating that the read count value exceeds the first read count limit, the stabilizing solution for use with subsequent biological samples, wherein the biological sample and the subsequent biological samples are of a same type.

Example 2 is the computer-implemented method of example 1, wherein the first sequencing range value corresponds to a fold-change value raised to a power that is based on an inverse of the logarithm of the enumerated abundance of the microbial species in the control sample.

Example 3 is the computer-implemented method of any of examples 1-2, wherein the stabilization framework includes a second read count limit defined based on the enumerated abundance of the microbial species in the control sample multiplied by a second sequencing range value.

Example 4 is the computer-implemented method of any of examples 1-3, wherein the second sequencing range value corresponds to a fold-change value raised to a negative power that is based on the inverse of the logarithm of the enumerated abundance of the microbial species in the control sample.

Example 5 is the computer-implemented method of any of examples 1-4, wherein the output of the stabilization framework is further based on whether the read count value is less than the first read count limit and greater than the second read count limit, and wherein selecting the stabilizing solution for use with subsequent biological samples is further in response to the output indicating that the read count value is less than the first read count limit and greater than the second read count limit.

Example 6 is the computer-implemented method of any of examples 1-5, wherein the mixture is stored for a predetermined time interval before the read count value is received.

Example 7 is the computer-implemented method of any of examples 1-6, wherein the mixture is exposed to one or more environmental conditions.

Example 8 is the computer-implemented method any of examples 1-7, wherein the read count value is derived by amplifying microbial deoxyribonucleic acid of the microbial species and determining a quantity of the microbial deoxyribonucleic acid.

Example 9 is the computer-implemented method any of examples 1-8, wherein the control sample and the biological sample are of a same type.

Example 10 is the computer-implemented method any of examples 1-9, wherein the first read count limit is derived by

$$A * {2}^{( 0.5 + \frac{5}{{log}_{10} ( A )} )} ,$$

wherein A corresponds to the read count of a particular microbial species in a baseline biological sample.

Example 11 is the computer-implemented method any of examples 1-10, wherein the second read count limit is derived by

$$A * {2}^{- ( 0.5 + \frac{5}{{log}_{10} ( A )} )} ,$$

wherein A corresponds to the read count of a particular microbial species in a baseline biological sample.

Example 12 is a system comprising: one or more processors; and a machine-readable storage medium storing instructions that when executed by the one or more processors, cause the one or more processors to perform any of example(s)s 1-11.

Example 13 is a machine-readable storage medium storing instructions that when executed by one or more processors, cause the one or more processors to perform any of example(s)s 1-11.

The disclosed system can be performed using a computing system. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device. The processor may be configured to carry out all or part of methods described herein for example by executing code for example stored in memory. One or more of a user device or computer, a provider server or system, or a suspended database update system may include the components of the computing system or variations on such a system.

This disclosure contemplates the computer system taking any suitable physical form. As example and not by way of limitation, the computer system may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh network of computing devices, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud, which may include one or more cloud components in one or more networks. Where appropriate, one or more computer systems may perform without substantial spatial or temporal limitation one or more steps of one or more methods described or illustrated herein. As an example, and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor may be, for example, be a conventional microprocessor such as an Intel Pentium microprocessor or Motorola power PC microprocessor. One of skill in the relevant art will recognize that the terms “machine-readable (storage) medium” or “computer-readable (storage) medium” include any type of device that is accessible by the processor. The memory can be coupled to the processor by, for example, a bus. The memory can include, by way of example but not limitation, random access memory (RAM), such as dynamic RAM (DRAM) and static RAM (SRAM). The memory can be local, remote, or distributed.

The bus can also couple the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer. The non-volatile storage can be local, remote, or distributed. The non-volatile memory is optional because systems can be created with all applicable data available in memory. A typical computer system will usually include at least a processor, memory, and a device (e.g., a bus) coupling the memory to the processor.

Software can be stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The bus can also couple the processor to the network interface device. The interface can include one or more of a modem or network interface. It will be appreciated that a modem or network interface can be considered to be part of the computer system. The interface can include an analog modem, Integrated Services Digital network (ISDN0 modem), cable modem, token ring interface, satellite transmission interface (e.g., “direct PC”), or other interfaces for coupling a computer system to other computer systems. The interface can include one or more input and/or output (I/O) devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device.

In operation, the computer system can be controlled by operating system software that includes a file management system, such as a disk operating system. One example of operating system software with associated file management system software is the family of operating systems known as Windows® from Microsoft Corporation of Redmond, WA, and their associated file management systems. Another example of operating system software with its associated file management system software is the Linux™ operating system and its associated file management system. The file management system can be stored in the non-volatile memory and/or drive unit and can cause the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “obtaining” or “forming” or “processing” or “computing” or “calculating” or“determining” or “displaying” or “generating” or the like, may refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

The algorithms and displays presented herein are not inherently related to any particular computer or other apparatus. Various general-purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any system capable of executing a set of instructions (sequential or otherwise) that specify actions to be taken by that system.

While the machine-readable medium or machine-readable storage medium is shown, by way of example, to be a single medium, the terms “computer readable medium”, “computer readable storage medium”, “machine-readable medium” and “machine-readable storage medium” should be taken to include a single medium or multiple media (e.g., a centralized or distributed database, and/or associated caches and servers) that store the one or more sets of instructions. The terms “computer readable medium”, “computer readable storage medium”, “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting the subject matter to the precise forms disclosed. Persons skilled in the relevant art can appreciate that many modifications and variations are possible in light of the above disclosure. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or.” in reference to a list of two or more items, covers all of the following interpretations of the word: any of the items in the list, all of the items in the list, or any combination of the items in the list.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

It is also noted that individual implementations may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps. A process may correspond to a method, a function, a procedure, a subroutine, a subprogram, etc. When a process corresponds to a function, its termination can correspond to a return of the function to the calling function or the main function.

Client devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things. The input devices can include, for example, a keyboard, a mouse, a keypad, a touch interface, a microphone, a camera, and/or other types of input devices. The output devices can include, for example, a display screen, a speaker, a haptic feedback system, a printer, and/or other types of output devices. A data storage device, such as a hard drive or flash memory, can enable the computing device to temporarily or permanently store data. A network interface, such as a wireless or wired interface, can enable the computing device to communicate with a network. Examples of computing devices include desktop computers, laptop computers, server computers, hand-held computers, tablets, smart phones, personal digital assistants, digital home assistants, as well as machines and apparatuses in which a computing device has been incorporated.

The various examples discussed above may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments). A processor(s), implemented in an integrated circuit, may perform the necessary tasks.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
generating, by a computing device, a stabilization framework usable to determine a viability of a stabilization solution to stabilize a particular microbial species, the stabilization framework including a first read count limit defined based on an enumerated abundance of the microbial species in a control sample multiplied by a first sequencing range value;
receiving a read count value corresponding to an enumerated abundance of the microbial species in a mixture comprising a biological sample and the stabilization solution;
applying the stabilization framework to the read count value, wherein the stabilization framework generates an output based on whether the read count value exceeds the first read count limit; and
selecting, in response to the output indicating that the read count value exceeds the first read count limit, the stabilizing solution for use with subsequent biological samples, wherein the biological sample and the subsequent biological samples are of a same type.

2. The computer-implemented method of claim 1, wherein the first sequencing range value corresponds to a fold-change value raised to a power that is based on an inverse of the logarithm of the enumerated abundance of the microbial species in the control sample.

3. The computer-implemented method of any of claims 1-2, wherein the stabilization framework includes a second read count limit defined based on the enumerated abundance of the microbial species in the control sample multiplied by a second sequencing range value.

4. The computer-implemented method of any of claims 1-3, wherein the second sequencing range value corresponds to a fold-change value raised to a negative power that is based on the inverse of the logarithm of the enumerated abundance of the microbial species in the control sample.

5. The computer-implemented method of any of claims 1-4, wherein the output of the stabilization framework is further based on whether the read count value is less than the first read count limit and greater than the second read count limit, and
wherein selecting the stabilizing solution for use with subsequent biological samples is further in response to the output indicating that the read count value is less than the first read count limit and greater than the second read count limit.

6. The computer-implemented method of any of claims 1-5, wherein the mixture is stored for a predetermined time interval before the read count value is received.

7. The computer-implemented method of any of claims 1-6, wherein the mixture is exposed to one or more environmental conditions.

8. The computer-implemented method any of claims 1-7, wherein the read count value is derived by amplifying microbial deoxyribonucleic acid of the microbial species and determining a quantity of the microbial deoxyribonucleic acid.

9. The computer-implemented method any of claims 1-8, wherein the control sample and the biological sample are of a same type.

10. The computer-implemented method any of claims 1-9, wherein the first read count limit is derived by
$$A * {2}^{( 0.5 + \frac{5}{{log}_{10} ( A )} )} ,$$
wherein A corresponds to the read count of a particular microbial species in a baseline biological sample.

11. The computer-implemented method any of claims 1-10, wherein the second read count limit is derived by
$$A * {2}^{- ( 0.5 + \frac{5}{{log}_{10} ( A )} )} ,$$
wherein A corresponds to the read count of a particular microbial species in a baseline biological sample.

12. A system comprising:
one or more processors; and
a machine-readable storage medium storing instructions that when executed by the one or more processors, cause the one or more processors to perform the methods of any of claims 1-11.

13. A machine-readable storage medium storing instructions that when executed by one or more processors, cause the one or more processors to perform the methods of any of claims 1-11.
